(19) United States
(12) Patent Application Publication
(10) Pub. No.: US 2022/0047249 A1
(43) Pub. Date: Feb. 17, 2022

(54) BIOLOGICAL DETECTION DEVICE AND METHOD FOR TOILET WATER SOLUTION

(71) Applicant: Taiwan RedEye Biomedical Inc., Hsinchu City (TW)

(72) Inventors: Shuo-Ting Yan, Hsinchu City (TW); I-Hua Wang, Hsinchu City (TW); Chen-Chung Chang, Hsinchu City (TW); Tsung-Jui Lin, Hsinchu City (TW)

(21) Appl. No.: 16/992,248

(22) Filed: Aug. 13, 2020

(51) Int. Cl. A61B 10/00; E03D 11/13; G01N 21/31; G01N 33/483; G01N 33/49; G01N 33/493

(52) U.S. Cl. CPC A61B 10/007; E03D 11/13; G01N 33/493; G01N 33/4833; G01N 33/49; G01N 21/31

(57) ABSTRACT

A biological detection device for toilet water solution includes a mounting portion, a box portion, an adjustable support portion and a detection portion. The box portion is connected to the mounting portion. The adjustable support portion can be adjusted for its movement in both horizontal and vertical directions and is connected to the mounting portion. The detection portion is connected and linked to the adjustable support portion and includes a light emitting source and an optical sensor. The biological detection device for toilet water solution is a home healthcare device capable of automatically detecting whether there is blood in excreta without adding any chemical reagent or collecting any excreta specimen.

### FIELD OF THE INVENTION

The present invention relates to a biological detection device and method, and more particularly to a biological detection device and method for toilet water solution.

### BACKGROUND OF THE INVENTION

The initial symptom of colorectal/rectal cancer is fecal occult blood, and the initial symptom of bladder cancer is urine occult blood, and both are related to occult blood. In the United States, the incidence of colorectal cancer accounts for 8% of the total incidence of cancers has a fourth rank in incidence rate and a second rank in mortality rate. In China, the incidence of colorectal cancer accounts 18.6% of the world, and the death toll accounts for 20.1% of the world, and both of the incidence and death toll ranks number one in the world. In Taiwan, the colorectal cancer is the top one of cancers in twelve consecutive years, and the incidence rate of colorectal cancer is the top one in the world. The journal “NATURE” presented in Nov. 9, 2017 that there was no universal screening method for bladder cancer. Only when people visually see blood in urine, they start knowing that there is a need for invasive endoscopic diagnosis and treatment, and approximately 10% of the patients having the symptom of blood in urine will be diagnosed with bladder cancer. It is feasible to use a non-invasive method for a urine test and can reduce the discomfort caused by the endoscopy.

A conventional immune occult blood test requires patients to collect a stool specimen by themselves, send the specimen to a hospital for testing, and wait for the test result. Such test is lack of immediate response and convenience. During the process of collecting the stool specimen, the occult blood in the stool is not uniformly distributed, occult blood may not be collected during the testing process to give a false negative result and cause a misjudgment. In some cases, a tumor or polyp bleeds intermittently, but not continuously, so that the tumor or polyp may not bleed on the same day or a day before collecting the stool specimen to give a false negative result and cause a misjudgment.

Japan Patent No. 1998339728 disclosed a stool composition examination device, wherein the device requires a stool collection step, a diluent addition step, and a washing step, and thus the use of such device is very inconvenient.

In addition, other conventional detection methods require adding chemicals that combine with the blood in the stool or urine, so that the additional step makes the test more inconvenient and the consumption of chemicals incurs a higher cost.

Therefore it is an important subject for related manufacturers to provide a device or method to meet the requirements of a quick and convenient biological detection while taking the cost and accuracy of the detection into consideration.

### SUMMARY OF THE INVENTION

In view of the aforementioned drawbacks of the conventional detection method, the present invention provides a biological detection device for toilet water solution to overcome the drawbacks of the prior art.

To achieve the aforementioned and other objectives, the present invention discloses a biological detection device toilet water solution provided for a biological detection, and the biological detection device comprises a mounting portion for mounting the biological detection device onto a toilet; a box portion including an operation control unit and coupled to the mounting portion; an adjustable support portion coupled to the mounting portion and capable of adjusting movements in a horizontal direction or a vertical direction and coupled to the mounting portion; and a detection portion placed into toilet water containing an excreta and comprising a light emitting source, and the detection portion being coupled and linked to the adjustable support portion, and the light emitting source being electrically coupled to the operation control unit; wherein, the operation control unit controls a light emitted from the light emitting source and passing through the toilet water containing an excreta to enter into an optical sensor, and after the optical sensor receives the penetrating light, a sensing signal being transmitted to the operation control unit, and the operation control unit determining whether or not there is a biological detection signal provided for outputting a detection result.

To achieve the aforementioned and other objectives, the present invention further discloses a biological detection method for toilet water solution, and the method is provided for a biological detection and applied to a biological detection device, and the biological detection device comprises a mounting portion, a box portion, an adjustable support portion and a detection portion, and the mounting portion of the device is mounted onto a toilet, and the box portion comprises an operation control unit and is coupled to the mounting portion, and the adjustable support portion is coupled to the mounting portion, and the detection portion is placed into toilet water containing an excreta and comprises a light emitting source, and the detection portion is coupled and linked to the adjustable support portion, and the light emitting source is electrically coupled to the operation control unit; and the biological detection method for toilet water solution comprises the steps of: linking the detection portion to the adjustable support portion in a horizontal direction or a vertical direction; receiving a start detection instruction from an input unit; driving the light emitting source to shoot out a light that passes through toilet water containing an excreta and enters into an optical sensor; and using the operation control unit to determine whether there is a biological detection signal, and transmitting a detection result to a display unit.

The present invention can achieve the aforementioned objectives of the biological detection and meet the requirements for convenience, comfortability, quickness, accuracy and cost-effectiveness.

### BRIEF DESCRIPTION OF THE DRAWINGS

FIG. 1 includes a front view and a right side view of a biological detection device for toilet water solution in accordance with a preferred embodiment of the present invention;

FIG. 2 is a schematic view of a biological detection device for toilet water solution installed to a toilet in accordance with a preferred embodiment of the present invention;

FIG. 3 is a schematic block diagram of a biological detection device for toilet water solution in accordance with a preferred embodiment of the present invention;

FIG. 4 is a schematic view showing the operating principle of a biological detection for toilet water solution in accordance with the present invention;

FIG. 5 is a graph of a blood absorption spectrum of a preferred embodiment of the present invention; and

FIG. 6 is a flow chart of a biological detection method for toilet water solution in accordance with a preferred embodiment of the present invention.

### DESCRIPTION OF THE PREFERRED EMBODIMENTS

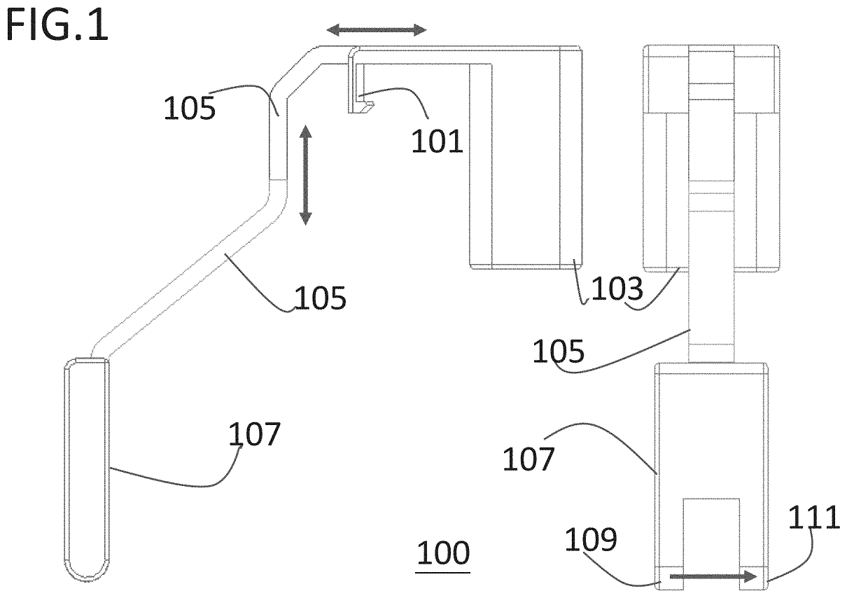

With reference to FIG. 1 for the front and right views of a biological detection device for toilet water solution in accordance with a preferred embodiment of the present invention, the biological detection device for toilet water solution 100 comprises a mounting portion 101, a box portion 103, an adjustable support portion 105 and a detection portion 107. The box portion 103 is coupled to the mounting portion 101. The adjustable support portion 105 can adjust its movements in both horizontal and vertical directions and is coupled to the mounting portion 101. The detection portion 107 is coupled and linked to the adjustable support portion 105. Wherein, the detection portion 107 comprises a light emitting source 109 and an optical sensor 111.

In another embodiment of the present invention, the adjustable support portion comprises a serrated structure, a telescopic sleeve, a universal shaft, a link rod or a keel structure, so that the adjustable support portion can be operated and adjusted by external forces within a specific range or controlled by internal electromechanical components to perform a movement, a twist, a slide, a displacement and/or a rotation in the X-axis, Y-axis, or Z-axis direction, after the detection portion coupled and linked to the adjustable support portion is submerged into the toilet water. It is noteworthy that the structure of the adjustable support portion of the present invention can be changed and is not limited by a particular structure.

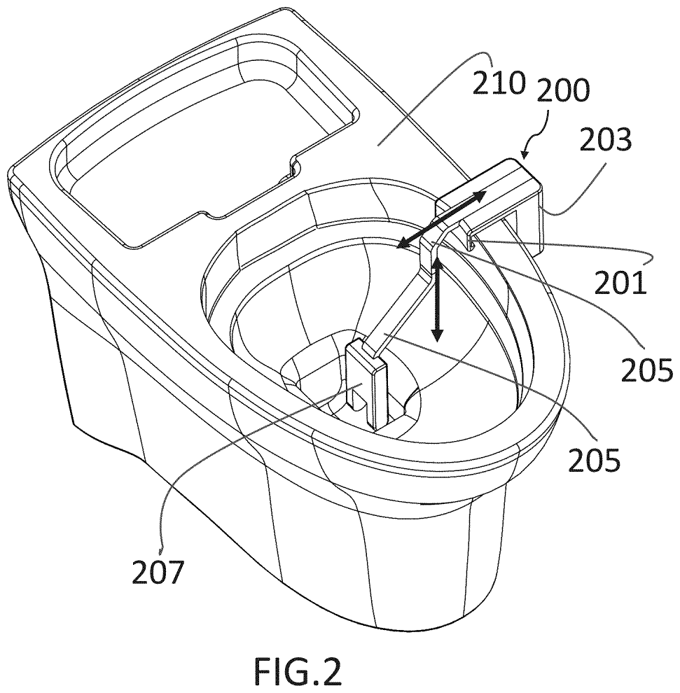

With reference to FIG. 2 for the schematic view of a biological detection device for toilet water solution installed to a toilet in accordance with a preferred embodiment of the present invention, the biological detection device for toilet water solution 200 comprises a mounting portion 201, a box portion 203, an adjustable support portion 205 and a detection portion 207. The mounting portion 201 is provided to mount the biological detection device for toilet water solution 200 onto a toilet 210. In a preferred embodiment, the biological detection device for toilet water solution 200 is mounted onto the toilet 210 by using the mounting portion 201 and a clamping method. The adjustable support portion 205 can be moved in both vertical and horizontal directions and linked to the detection portion 207. The detection portion 207 is placed into toilet water (not shown in the figure) containing an excreta (urine and/or stool) in the toilet 210, and the detection portion 207 is linked by the adjustable support portion 205 and can be adjusted and then fixed to an appropriate detection position in the toilet water containing an excreta in the toilet 210.

In another embodiment of the present invention, the mounting portion is used to mount, fix, or install the biological detection device for toilet water solution to a cover of a toilet or a side of the cover by a clamping, suspending, adsorbing, pasting, binding, socketing, latching buckling, magnetically attracting, riveting, screwing, or locking method, and the corresponding devices used in the aforementioned methods include a clamp, an adhesive, a strap, a screw, a nut, a snap unit, a magnet, a rivet, or a sucker, etc., wherein the form of the mounting portion is not limited specifically.

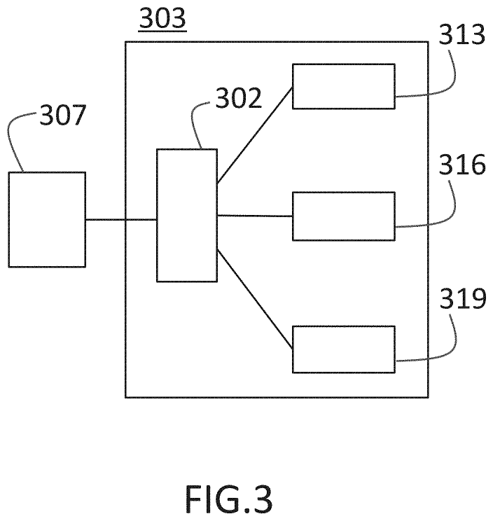

With reference to FIG. 3 for the schematic block diagram of a biological detection device for toilet water solution in accordance with a preferred embodiment of the present invention, the box portion 303 comprises an operation control unit 302, an input unit 313, a display unit 316 and a power supply 319. The operation control unit 302 is electrically coupled to the input unit 313, the display unit 316 and the power supply 319, and the operation control unit 302 is electrically coupled to the light emitting source and the optical sensor of the detection portion 307.

In the present invention, the operation control unit such as a MCU or a CPU is a unit with the computing, processing, controlling and/or analyzing functions. The power supply includes a battery (a replaceable battery or a rechargeable battery) or an external alternate current. The input unit such as a physical button, a voice control module or an LCD touch display device is provided for receiving a detection instruction or an adjustable support portion control instruction. If the input unit is the LCD touch display device, the input unit may further include a user interface. The input unit has different options for different detecting targets (such as blood, uric acid, urine protein, and urine sugar). The display unit such as the LCD touch display device or LED lights is provided for displaying detection results and information. The light emitting source such as an LED light source or a laser has a controller for emitting light beams of different wavelengths. The optical sensor such as a spectrophotometer, a photo-diode, a complementary metal oxide semiconductor (CMOS) sensor, or a charge coupled device (CCD) sensor has a controller. It is noteworthy that this invention is not limited by the aforementioned embodiments.

In another embodiment, the box portion can be coupled to an external washlet wherein the washlet may even have the power supply. Further, the box portion can be integrated with the washlet, so that the box portion and the washlet share the operation control unit, the input unit, the display unit, and the power supply.

The biological detection device for toilet water solution of the present invention is used for a biological detection. In a preferred embodiment of the present invention, the biological detection of blood is used for illustrating the invention. In other embodiments, the biological detection may be a detection of uric acid, urine protein or urine sugar. In a different implementation, the principle of the biological detection is to make use of the properties of absorption, fluorescence, scattering, or Raman spectrum. However, the invention is not limited to the aforementioned embodiments only.

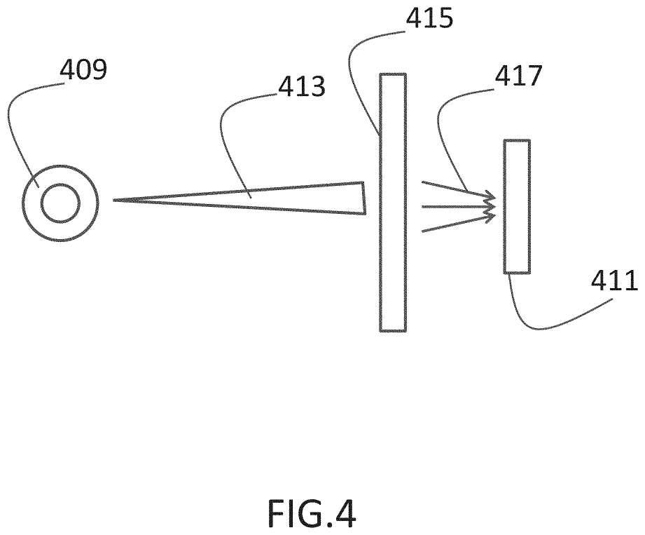

With reference to FIG. 4 for the schematic view showing the principle of a biological detection method for toilet water solution in accordance with the present invention, the operation control unit controls a light emitting source 409 to emit a light beam 413 with a specific wavelength (such as 250˜650 nm), and the light beam 413 enters and passes through the toilet water containing an excreta 415 (which is an object to be tested) to become a penetrating light 417 which enters into an optical sensor 411. After receiving the penetrating light 417, the optical sensor 411 transmits a sensing signal to the operation control unit.

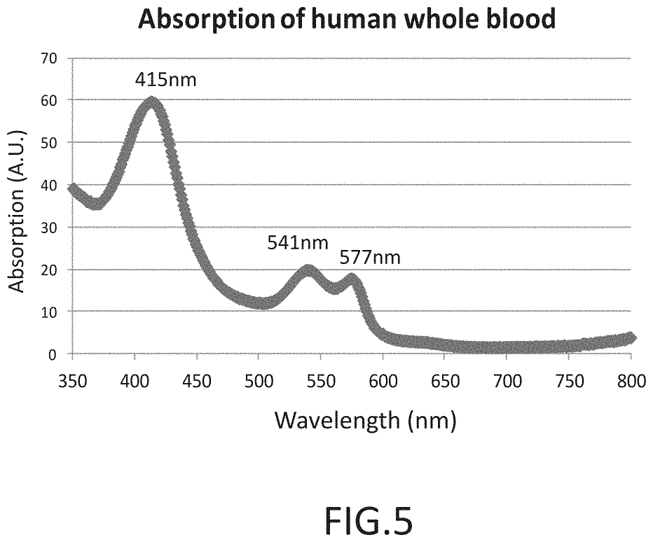

With reference to FIG. 5 for a blood absorption spectrum of a preferred embodiment of the present invention, the operation control unit determines whether or not there is a biological detection signal (which is an occult blood signal in this embodiment of the invention) after receiving the sensing signal. If the operation control unit determines that there is a sensing signal with characteristic absorption peaks of approximately 415 nm, 541 nm or 577 nm as shown in FIG. 5, then the sensing signal will be determined as an occult blood (hemoglobin) signal. The operation control unit outputs a detection result (with the occult blood signal) to a display unit and controls the display unit to display the detection result. In another embodiment, the light beam emitted from a light source may have a different wavelength according to a different the target of the biological detection (such as uric acid, urine protein or urine sugar), and the spectral determination method may be different (according to the clinical trial results and statistical data.).

The foregoing preferred embodiment uses biophysics of the blood for the detection. In addition to the detection of a trace amount of blood, the biological detection device for toilet water solution of the invention can also be used in other biological detections such as those for uric acid, urine protein, and urine sugar.

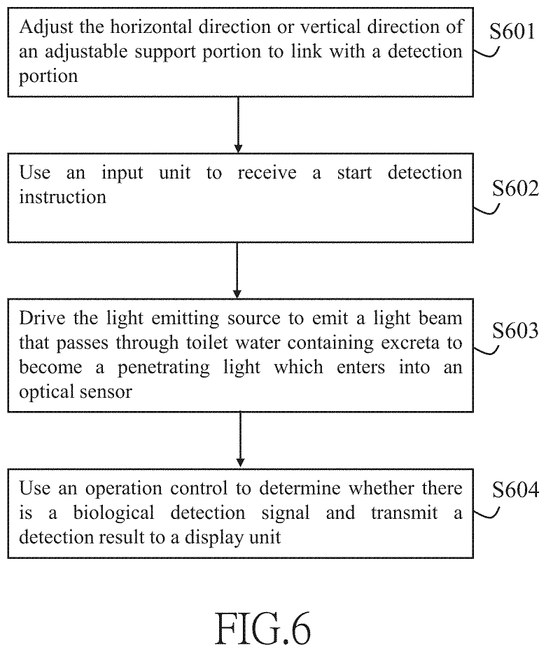

With reference to FIG. 6 for the flow chart of a biological detection method for toilet water solution in accordance with an embodiment of the present invention, the biological detection method is used for a biological detection, and this embodiment uses a biological detection for blood to illustrate the invention. In other embodiments, a biological detection for uric acid, urine protein or urine sugar can be performed, and the principle of the biological detections in other embodiments makes use of the properties of absorption, fluorescence, scattering, or Raman spectrum. It is noteworthy that the invention is not limited to these embodiments only.

The biological detection method of the present invention is applied to a biological detection device for toilet water solution. The related contents of the biological detection device for toilet water solution in accordance with a preferred embodiment of the present invention have been described above and illustrated by FIGS. 1, 2 and 3. The biological detection device for toilet water solution comprises a mounting portion, a box portion, an adjustable support portion and a detection portion. The mounting portion is provided for mounting the biological detection device for toilet water solution onto a toilet. The adjustable support portion can be moved in both vertical and horizontal directions and linked to the detection portion. The detection portion is placed into toilet water containing excreta (urine and/or stool) in the toilet, and the detection portion is linked by the adjustable support portion and can be adjusted to an appropriate detection position in the toilet water containing an excreta in the toilet. The box portion comprises an operation control unit, a display unit, an input unit and a power supply and is coupled to the mounting portion. The detection portion comprises a light emitting source and an optical sensor, and the light emitting source and the optical sensor are electrically coupled to the operation control unit.

In an embodiment of the present invention, the biological detection method for toilet water solution comprises the following steps:

Step S601: The horizontal direction and/or vertical direction of an adjustable support portion to link with a detection portion is adjusted, wherein the movement in the X-axis, Y-axis and Z-axis directions, twisting, sliding, displacement and/or rotation of the adjustable support portion is adjusted and operated by an external force or by an internal electromechanical control, and the detection portion coupled and linked to the adjustable support portion is placed at an appropriate detection position in toilet water and then fixing at the appropriate detection position.

Step S602: An input unit is used to receive a start detection instruction, wherein the input unit is used to receive a start detection instruction inputted by a user, so as to start the detection, and the input unit such as a physical button, a sound control module, or an LCD touch display device is provided for the user to enter the start detection instruction or control the adjustable support portion to perform the adjusting instruction, and if the input unit is the LCD touch display device, then the input unit can include a user interface, and the input unit has different options for different detecting targets (such as blood, uric acid, urine protein and urine sugar). It is noteworthy that the sequence of the steps S601 and S602 is not limited.

Step S603: The light emitting source is driven to emit a light beam that passes through toilet water containing excreta to become a penetrating light which enters into an optical sensor.

With reference to FIG. 4 and the above description related to FIG. 4, the input unit is provided for receiving a start detection instruction to generate a start signal, so that the operation control unit controls a light emitting source 409 to emit a light beam 413 with a specific wavelength (such as 250˜650 nm) and enters and passes the light beam 413 into the toilet water containing an excreta 415 (object to be tested) to become a penetrating light 417 which enters into an optical sensor 411, and after the optical sensor 411 receives the penetrating light 417, the optical sensor 411 transmits the sensing signal to the operation control unit.

Step S604: The operation control unit is provided to determine whether there is a biological detection signal and transmits a detection result to a display unit.

With reference to FIG. 5 and the above description related to FIG. 5, after the operation control unit receives the sensing signal, the operation control unit determines whether there is a biological detection signal (such as an occult blood signal in this embodiment). If the sensing signal is determined to be having characteristic peak values of appropriately 415 nm, 541 nm or 577 nm as shown in FIG. 5, then the sensing signal will be determined as an occult blood (hemoglobin) signal. The operation control unit outputs a detection result (with the occult blood signal) to a display unit and controls the display unit to display the detection result. In another embodiment, the light beam emitted from a light source may have a different wavelength according to a different the target of the biological detection (such as uric acid, urine protein or urine sugar), and the spectral determination method may be different (according to the clinical trial results and statistical data.).

In addition, the mounting portion is used to mount, fix or install the biological detection device for toilet water solution onto a toilet by a clamping, suspending, adsorbing, pasting, binding, socketing, latching buckling, magnetically attracting, riveting, screwing, or locking method, and the corresponding devices used in the aforementioned methods include a clamp, an adhesive, a strap, a screw, a nut, a snap unit, a magnet, a rivet, or a sucker, etc., wherein the form of the mounting portion is not limited specifically.

In another embodiment, the adjustable support portion can include a serrated structure, a telescopic sleeve, a universal shaft, a link rod or a keel structure, so that the movement in the X-axis, Y-axis and Z-axis directions, twisting, sliding, displacement and/or rotation of the adjustable support portion is adjusted and operated by an external force or by an internal electromechanical control, and the detection portion coupled and linked to the adjustable support portion is placed at an appropriate detection position in toilet water and then fixing at the appropriate detection position.

In another embodiment, the box portion is combined with an external washlet, wherein the washlet even has a power supply. Further, the box portion can be integrated with the washlet to share the operation control unit, the input unit, the display unit and the power supply.

In the basic principle of the aforementioned detection method in accordance with an embodiment of the present invention, the blood absorption spectrum is measured. In fact, it should not be limited to the blood absorption only, but the properties of fluorescence, scattering, and Raman spectrum generally provided for measuring blood can be used as a basis to determine whether or not the toilet water containing the excreta has a trace amount of blood.

In summation of the description above, the present invention provides a biological detection device for toilet water solution (which is a home healthcare device) and a biological detection method for toilet water solution to automatically detect whether or not the excreta contains blood, uric acid, urine protein or urine sugar without requiring the addition of any biological/chemical reagents, diluents, and test paper, and without requiring the collection of any excreta specimen or any stool or urine collection procedure. Therefore, the device and method are suitable for homecare operations. A non-invasive method is used to detect whether or not the excreta is normal, and this method can reduce the discomfort caused by the conventional endoscopic examination and achieve the effect of the biological detection of the present invention

While the invention has been described by means of specific embodiments, numerous modifications and variations could be made thereto by those skilled in the art without departing from the scope and spirit of the invention as set forth in the claims.

What is claimed is:

1. A biological detection device for toilet water solution, provided for a biological detection, comprising:
a mounting portion, for mounting the biological detection device onto a toilet;
a box portion, including an operation control unit, and coupled to the mounting portion;
an adjustable support portion, coupled to the mounting portion; and
a detection portion, placed into toilet water containing an excreta, and comprising a light emitting source, and the detection portion being coupled and linked to the adjustable support portion, and the light emitting source being electrically coupled to the operation control unit;
wherein, the operation control unit controls a light emitted from the light emitting source and passing through the toilet water containing the excreta to enter into an optical sensor, and after the optical sensor receives the penetrating light, a sensing signal being transmitted to the operation control unit, and the operation control unit determining whether or not there is a biological detection signal provided for outputting a detection result.

2. The biological detection device for toilet water solution as claimed in claim 1, wherein the box portion further comprises an input unit and a display unit electrically coupled to the operation control unit, and the input unit is provided for inputting a detection instruction, and the operation control unit controls the display unit to display the detection result.

3. The biological detection device for toilet water solution as claimed in claim 1, wherein the optical sensor is included in the detection portion.

4. The biological detection device for toilet water solution as claimed in claim 1, wherein the biological detection is a detection of blood, uric acid, urine protein or urine sugar.

5. The biological detection device for toilet water solution as claimed in claim 1, wherein the biological detection adopts a detection principle of using the properties of absorption, fluorescence, scattering, or Raman spectrum.

6. The biological detection device for toilet water solution as claimed in claim 1, wherein the mounting portion is used to mount the biological detection device for toilet water solution onto a toilet by a clamping, suspending, adsorbing, pasting, binding, socketing, latching buckling, magnetically attracting, riveting, screwing, or locking method.

7. The biological detection device for toilet water solution as claimed in claim 1, wherein the adjustable support portion comprises a serrated structure, a telescopic sleeve, a universal shaft, a link rod or a keel structure.

8. The biological detection device for toilet water solution as claimed in claim 1, wherein the box portion is combined with a washlet.

9. A biological detection method for toilet water solution, provided for a biological detection and applied to a biological detection device, and the biological detection device comprising a mounting portion, a box portion, an adjustable support portion and a detection portion, and the mounting portion of the device being mounted onto a toilet, and the box portion comprising an operation control unit and being coupled to the mounting portion, and the adjustable support portion being coupled to the mounting portion, and the detection portion being placed into toilet water containing an excreta and comprising a light emitting source, and the detection portion being coupled and linked to the adjustable support portion, and the light emitting source being electrically coupled to the operation control unit; and the biological detection method comprising the steps of:
linking the detection portion to the adjustable support portion in a horizontal direction or a vertical direction;
receiving a start detection instruction from an input unit;
driving the light emitting source to shoot out a light that passes through toilet water containing the excreta and enters into an optical sensor; and
using the operation control unit to determine whether there is a biological detection signal, and transmitting a detection result to a display unit.

10. The biological detection method for toilet water solution as claimed in claim 9, wherein the box portion further comprises the input unit and the display unit electrically coupled to the operation control unit.

11. The biological detection method for toilet water solution as claimed in claim 9, wherein the optical sensor is included in the detection portion.

12. The biological detection method for toilet water solution as claimed in claim 9, wherein the biological detection is a detection of blood, uric acid, urine protein or urine sugar.

13. The biological detection method for toilet water solution as claimed in claim 9, wherein the biological detection adopts a detection principle of using the properties of an absorption, fluorescence, scattering or Raman spectrum.

14. The biological detection method for toilet water solution as claimed in claim 9, wherein the biological detection device for toilet water solution is mounted onto a toilet by a clamping, suspending, adsorbing, pasting, binding, socketing, latching buckling, magnetically attracting, riveting, screwing, or locking method.

15. The biological detection method for toilet water solution as claimed in claim 9, wherein the adjustable support portion comprises a serrated structure, a telescopic sleeve, a universal shaft, a link rod or a keel structure.

16. The biological detection method for toilet water solution as claimed in claim 9, wherein the box portion is combined with a washlet.
